(19) United States
(12) Patent Application Publication
(10) Pub. No.: US 2002/0147330 A1
(43) Pub. Date: Oct. 10, 2002

(54) Probe carrier, method of manufacturing the same and apparatus to be used for the method

(76) Inventors: Nobuko Yamamoto, Kanagawa (JP); Naoto Mihashi, Tokyo (JP); Hiroto Yoshii, Tokyo (JP)

Correspondence Address: FITZPATRICK CELLA HARPER & SCINTO, 30 ROCKEFELLER PLAZA, NEW YORK, NY, 10112, US

(21) Appl. No.: 10/106,460

(22) Filed: Mar. 27, 2002

(30) Foreign Application Priority Data
Mar. 28, 2001 (JP) 093267/2001(PAT.)

(51) Int. Cl. C07H 21/04

(52) U.S. Cl. 536/24.32

(57) ABSTRACT

A probe carrier of a DNA array or the like is formed by arranging regions fixing different probes in the axial direction of a substrate having an oblong profile which is string-shaped, tape-shaped, string-shaped, tube-shaped or rod-shaped. With this arrangement, the efficiency of manufacturing a probe carrier and handling it in different steps of specimen analysis is improved.

### BACKGROUND OF THE INVENTION

1. Field of the Invention

This invention relates to a probe carrier carrying probes rigidly secured thereto for detecting a target substance and a method and an apparatus for detecting a target substance, using such a probe carrier. The present invention also relates to a method and an apparatus for manufacturing such a probe carrier.

2. Related Background Art

When analyzing the base sequence of a gene DNA or conducting a gene diagnosis by analyzing a number of items simultaneously, a number of different probe species are needed to single out a DNA having a target base sequence in order to raise the reliability of operation. DNA microchips have been attracting attention as means for providing a number of different probe species to be used for such sorting operations. A large number of solution specimens (e.g., 96, 384 or 1,536 specimens) containing proteins or drugs to be sorted normally have to be subjected to a screening operation in an orderly manner in the field of high throughput screening of chemicals or combinatorial chemistry. For these purposes, techniques of sequentially arranging a large number of different drugs, automatic screening technologies and dedicated devices for sorting the drugs arranged in this way and software for controlling a series of screening operations and statistically processing the obtained results have been and being developed.

Basically, such screening operations as described above that are conducted in parallel simultaneously consist in detecting an action or non-action or a response or non-response of each specimen to known probes arranged in array, or a probe array, provided as means for sorting the substances in specimens for evaluation under same conditions. Generally, the action or response to be used with each probe is defined in advance and therefore substances of a same type are normally used as probe species that are mounted on a probe array. Then, the probe array may be an array of DNA probes carrying a group of DNAs having different respective base sequences. DNAs, proteins and synthesized chemicals (drugs) are examples of substances that may be used for a group of probes. While a probe array of a plurality of different probe species is used in many instances, a large number of identical DNAs having a same base sequence, identical proteins having a same amino acid sequence or identical chemical substances may be arranged in array depending on the type of screening operation. Such probes are mainly used for screening drugs.

In a probe array formed from a plurality of different probe species, a group of DNAs having different base sequences, a group of proteins having different amino acid sequences, a group of different chemical substances or the like are often arranged in array on a substrate according to a predetermined sequence of arrangement. Particularly, DNA probe arrays are used for analyzing the base sequence of a gene DNA or conducting a gene diagnosis by analyzing a number of items simultaneously in order to raise the reliability of operation as pointed out above.

U.S. Pat. No. 5,424,186 describes a technique of preparing an array of DNA probes with DNAs having respective base sequences that are different from each other by means of stepwise elongation reaction conducted on a carrier by utilizing photodecomposable protective groups and photolithography. With the proposed technique, it is possible to prepare a DNA probe array carrying DNAs of more than 10,000 different kinds that are different from each other in terms of base sequence per 1 cm. The process of synthesizing DNAs by means of stepwise elongation reaction, used in this technique, comprises a photolithography step in which dedicated photomasks are used respectively for the four different kinds of base (A, T, C, G) in order to selectively elongate any of the bases at a predetermined position of the array so that consequently DNAs of different species having desired respective base sequences are synthetically produced and arranged on a substrate in a predetermined order.

Beside the above described technique, techniques of manufacturing a probe array by synthesizing DNAs for probes in advance in a refined manner, confirming, if necessary, their respective base lengths and supplying the DNAs to a substrate by means of an appropriate device such as a microdispenser are known. PCT Patent Publication WO95/35505 describes a technique of supplying DNAs onto a membrane by means of a capillary. With this technique, it is theoretically possible to manufacture an array of about 1,000 DNAs per 1 cm². It is basically a technique of manufacturing a probe array by supplying a probe solution to a predetermined position of a substrate for each probe by means of a capillary-shaped dispensing device and repeating this operation.

There are also known techniques of supplying a solution of a substance necessary for conducting an operation of DNA solid phase synthesis on a substrate by using an ink-jet method in each elongation step. For example, European Patent Publication EP0703825B1 describes a technique of synthesizing DNAs of a plurality of different kinds having respective predetermined base sequences in a solid phase by supplying nucleotide monomers and activators by means of respective piezo jet nozzles for the purpose of solid phase synthesis of DNAs. This supply (application) technique utilizing an ink-jet method is reliable in terms of reproducibility of supply rate if compared with a solution supply (application) technique utilizing a capillary and also provides advantages for realizing high density probe arrays because the nozzle structure of the ink-jet system can be miniaturized.

Japanese Patent Application Laid-open No.11187999 discloses a method of forming a spot containing a probe by causing a droplet of liquid containing the probe to adhere to a solid phase by means of a bubble-jet head.

### SUMMARY OF THE INVENTION

In view of the above identified circumstances, it is therefore an object of the present invention to provide a novel type of probe carrier carrying a DNA array or the like that can be manufactured at low cost and handled easily and efficiently in the manufacturing process and in many different steps of analyzing specimens. Another object of the present invention is to provide a method of manufacturing such a novel type of probe carrier and a method of detecting a target substance by using such a probe carrier.

In an aspect of the invention, there is provided a probe carrier having on an elongate substrate a plurality of regions fixing different probes each adapted to be bonded specifically to a target substance, the regions fixing probes being arranged longitudinally on the substrate.

In another aspect of the invention, there is provided a cassette for detecting a target substance by means of probes adapted to be bonded specifically to a target substance, the cassette comprising in a cabinet:

a probe carrier roll containing section containing a probe carrier roll formed by winding on a feed reel a probe carrier having on an elongate substrate a plurality of regions fixing different probes each adapted to be bonded specifically to a target substance, the regions fixing probes being arranged longitudinally on the substrate;

a take-up reel for taking up the probe carrier fed out from the feed reel; and

an opening arranged on the moving path of the probe carrier from the probe carrier roll containing section to the take-up reel.

In still another aspect of the invention, there is provided a method for detecting a target substance in a specimen by means of probes adapted to be bonded specifically to the target substance, the method comprising:

a step of causing a probe carrier having regions fixing different probes to contact with the specimen, the regions being arranged in the longitudinal direction of an elongate substrate; and

a step of detecting presence or absence of a reaction of the target substance with the probe of each of the regions fixing probes caused to contact with the specimen.

In still another aspect of the invention, there is provided a target substance detecting apparatus for detecting presence or absence of a reaction of probes with a target substance in a specimen by detecting a signal generated by the reaction, the probes being adapted to be bonded specifically to the target substance, the apparatus comprising:

a signal detecting means; and

a moving means for longitudinally moving a sample for observation relative to the detecting means, the sample for observation being obtained by causing the specimen to contact with a probe carrier having regions fixing different probes, the regions being arranged in the longitudinal direction of an elongate substrate.

In still another aspect of the invention, there is provided a method for detecting a target substance in a specimen by means of probes adapted to be bonded specifically to the target substance, the method comprising:

(1) a step of preparing a probe carrier by arranging regions fixing different probes in the longitudinal direction of an elongate substrate;

(2) a step of obtaining a sample for observation by causing the probe carrier to contact with the specimen: and

(3) a step of detecting presence or absence of a reaction of the target substance with the probe in each of the regions fixing probes in the sample for observation.

In still another aspect of the invention, there is provided a detection apparatus for detecting a reaction of probes adapted to be bonded specifically to a target substance with the target substance in a specimen by detecting presence or absence of a signal generated by the reaction, the apparatus comprising:

a probe carrier preparing section for preparing a probe carrier carrying probes fixed thereto on an elongate substrate;

a sample preparing section for preparing a sample for observation by causing the probe carrier prepared by the probe carrier preparing section to contact with a specimen; and

a detecting section for detecting a signal generated by the reaction of the probes with a target substance in the sample for observation as prepared by the sample preparing section;

the probe carrier preparing section having:

a means for applying solutions of the probes;

a means for holding the substrate; and

a first moving means for moving the solution applying means longitudinally relative to the substrate;

the detecting section having:

a means for detecting the signal; and

a second moving means for moving the signal detecting means relative to the sample for observation as prepared by causing the probe carrier having regions fixing different probes to contact with the specimen, the regions being arranged in the longitudinal direction of the substrate.

In still another aspect of the invention, there is provided a method for manufacturing a probe carrier having regions fixing different probes adapted to be bonded specifically to a target substance, the regions being arranged in the longitudinal direction of an elongate substrate, the method comprising:

a step of moving a means for applying probe solutions relative to the substrate from the front end to the rear end thereof so as to apply different probe solutions as a function of the arrangement of the regions; and

a step of fixing the probes in the regions carrying the respective probe solutions applied thereto to the substrate.

In still another aspect of the invention, there is provided a probe carrier manufacturing apparatus for manufacturing a probe carrier having regions fixing different probes adapted to be bonded specifically to a target substance, the regions being arranged in the longitudinal direction of an elongate substrate, the apparatus comprising:

a means for applying probe solutions; and

a means for relatively moving the solution applying means from the front end to the rear end of the long substrate.

In still another aspect of the invention, there is provided a probe carrier comprising:

a plurality of regions fixing probes arranged axially in the inside of a tubular member, the regions carrying different probes fixed thereto.

In still another aspect of the invention, there is provided a method for detecting a target substance in a specimen by means of probes adapted to be bonded specifically to the target substance, the method comprising:

a step of flowing a solution containing the specimen from one of two openings of a tubular member as a probe carrier to the other opening, a plurality of regions fixing probes being arranged axially in the inside of the tubular member, the regions carrying different probes fixed thereto; and

a step of detecting presence or absence of a reaction of the target substance with the probe of each of the regions fixing probes caused to contact with the specimen.

In still another aspect of the invention, there is provided a method of manufacturing a probe carrier having a plurality of regions fixing probes adapted to be bonded specifically to a target substance, the regions being arranged axially in the inside of a tubular member, the regions carrying different probes fixed thereto, the method comprising:

a step of causing solutions of a plurality of probes necessary for forming and arranging the regions to adhere to a plate-shaped member according to the arrangement of the regions;

a step of solidifying the probes in the regions carrying the probes adhered thereto on the plate-shaped member; and

a step of molding the plate-shaped member carrying the solidified probes adhered thereto into a tubular member so as to make the regions fixing probes to be arranged in the axial direction on the inner wall of the tubular member.

In a further aspect of the invention, there is provided a method for detecting a target substance in a specimen by means of probes adapted to be bonded specifically to a target substance, the method comprising:

a step of flowing a solution containing the specimen into the inside of a tubular member having regions fixing probes arranged in the axial direction in the inside thereof and causing the solution to contact with each of the regions fixing probes to obtain a sample for observation; and

a step of detecting presence or absence of a reaction of the target substance with the probe of each of the regions fixing probes in the sample for observation.

Thus, according to the invention, there is provided a novel type of probe carrier carrying a DNA array or the like that can be manufactured at low cost and handled easily and efficiently in the manufacturing process and in many different steps of analyzing specimens. According to the invention, there are also provided a method of manufacturing such a novel type of probe carrier and a method of detecting a target substance by using such a probe carrier as well as an apparatus to be used for such methods.

### BRIEF DESCRIPTION OF THE DRAWINGS

FIGS. 1A and 1B are schematic illustrations of oblong probe carriers, showing their structures.

FIG. 2 is a schematic illustration of a step of operation of applying liquid containing probes onto a string-shaped substrate from a liquid-jet apparatus and arranging regions for fixing probes.

FIG. 3 is a schematic illustration of a method of manufacturing a probe carrier by supplying a substrate in the form of a roll.

FIG. 4 is a schematic illustration of a method of manufacturing a probe carrier by supplying a substrate contained in a cassette.

FIGS. 5A, 5B and 5C are schematic illustrations of methods of causing a long probe carrier to contact with a specimen, of which FIG. 5A shows an immersion method, FIG. 5B shows a method of causing a substrate to continuously pass through a solution containing a specimen and FIG. 5C shows a method of applying liquid containing a specimen by means of a liquid-ejecting apparatus.

FIGS. 6A, 6B and 6C are schematic illustrations of a step of detecting signals from respective labels.

FIG. 7 is a schematic illustration of a step of manufacturing a long probe carrier.

FIG. 8 is a schematic illustration of a liquid-ejecting apparatus, showing its configuration.

FIGS. 9A and 9B are schematic illustrations of a probe carrier utilizing the inside of a tubular member.

FIGS. 10A and 10B are schematic illustrations of a probe carrier, showing the internal structure of a tubular member formed by utilizing a groove formed on the surface of a plate-shaped member.

FIG. 11 is a schematic illustration of regions fixing probes to be transferred onto a rod.

### DESCRIPTION OF THE PREFERRED EMBODIMENTS

As far as this specification is concerned, the probes that are fixed to a probe carrier can be specifically bonded to a specific target substance. The probes may include oligonucleotides, polynucleotides and/or other polymers that can be recognized by a specific target. The term “probe” as used herein refers to a molecule that can operate as probe such as a polynucleotide molecule or a group of molecules having a same probe function such as polynucleotide molecules that are fixed to distributed respective positions of a surface and have a same sequence and includes molecules called ligands. The term “probe” and the term “target” can often be used exchangeably and a probe can be a substance capable of being bonded or made to become bonded to a target as part of a ligand-antiligand (which may also be referred to as receptor) pair. For the purpose of the invention, a probe and a target can include naturally occurring bases and/or analogs.

Examples of such a probe as can be supported on a carrier include a substance with a structure having a bonding section by way of a linker in a part of an oligonucleotide having a base sequence that can be hybridized with a target nucleic acid, the bonding section being to be bonded to the surface of the carrier. There are no limitations to the position of the bonding section to be bonded to the carrier in the oligonucleotide molecule so long as the desired reaction of hybridization is not adversely affected.

While the probes to be used for a probe array for the purpose of the invention may be appropriately selected depending on the application purpose thereof, it is preferable that one or more than one types of probes are selected from DNAs, RNAs, cDNAs (complementary DNAs), PNAs, oligonucleotides, polynucleotides, other nucleic acids, oligopeptides, polypeptides, proteins, enzymes, substrates relative to enzymes, antibodies, epitopes relative to antibodies, antigens, hormones, hormone receptors, ligands, ligand receptors, oligosaccharides and polysaccharides for the purpose of advantageously embodying the present invention.

For the purpose of the invention, a probe carrier refers to an object realized by fixing a plurality of probe species in independent respective regions of the surface of a carrier (including the surface of the inner walls of hollow members or tubular carrier members) typically as dot-shaped spots, while a probe array refers to a probe carrier where those probe species are arranged at a predetermined interval.

Each probe has a structure that makes it possible to be bonded to the surface of a carrier and is preferably bonded onto the carrier by way of the structure that makes it possible to be bonded to the surface thereof. Preferably, the structure of each probe that makes it possible to be bonded to the surface of the carrier is formed by means of a process of introducing at least one organic functional group species such as amino group, mercapto group, carboxyl group, hydroxyl group, acid halide (haloformyl group; -COX), halide (-X), aziridine, maleimide group, succinimide group, isothiocyanate group, sulfonylchloride group (—SO₂Cl), aldehyde group (formyl group; —CHO), hydrazine or acetamide iodide. Whenever necessary, the surface of the carrier may be treated appropriately in a manner that depends on the structure on the probe side necessary for bonding the probe to the carrier.

Now, the present invention will be described by referring to the accompanying drawings that illustrate preferred embodiments of the invention. (Long probe carrier and utilization thereof)

A long probe carrier according to the invention has two or more than two regions that are arranged longitudinally on a substrate having an oblong profile and carry different probes. A long probe carrier according to the invention may be thread-shaped, tape-shaped (belt-shaped), string-shaped, tube-shaped or rod-shaped. A long substrate on which a long probe carrier is formed may show a degree of rigidity that makes it either non-deformable or deformable or may not have any particular shape like a thread or a string. In other words, it may be selected from various objects depending on the application thereof. Such a substrate may be made of glass, paper or resin of any types.

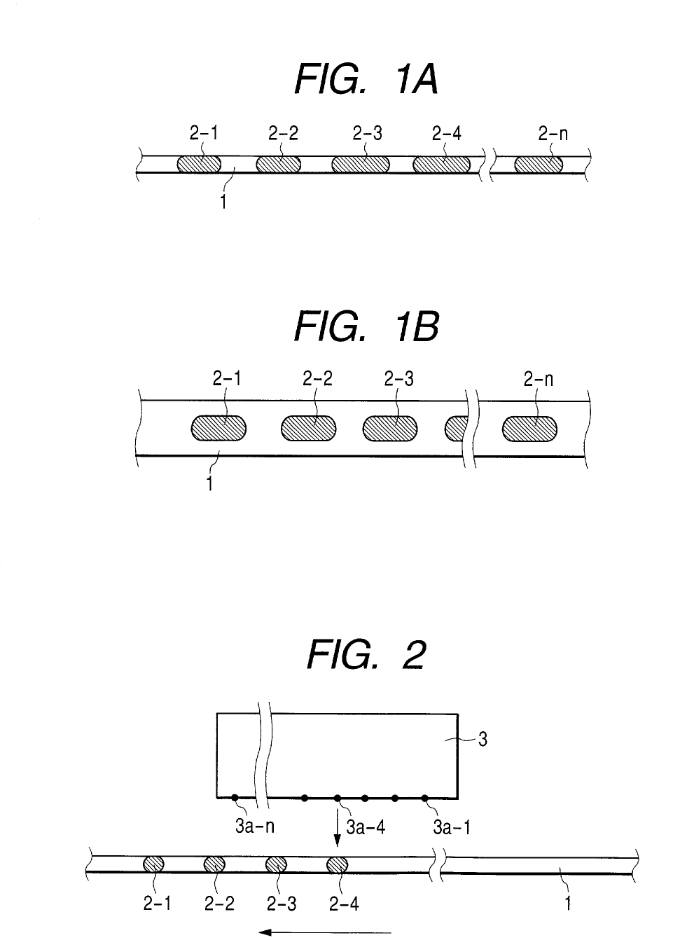

FIGS. 1A and 1B schematically illustrate embodiments of probe carrier according to the invention. FIG. 1A is a schematic illustration of part of a thread-shaped or string-shaped carrier 1 that has longitudinally (axially) arranged regions 2-1 through 2-n for fixing different probes.

FIG. 1B is a schematic illustration of part of a tape-shaped substrate 1 that has regions 2-1 through 2-n fixing different probes. The regions fixing probes formed on the tape-shaped substrate may be arranged on only one of the surfaces thereof or on the opposite surfaces thereof depending on the material of the substrate. Alternatively, each of the regions may be formed in such a way that the region formed on one of the surfaces permeates to the other surface. The probe carrier formed by using such a tape-shaped carrier may show a degree of rigidity that makes it able to maintain its original shape or may be deformable.

On the other hand, a long probe carrier according to the invention may be provided as a piece of thread, string, tape or chip that is cut to a predetermined length. Still alternatively, it may be provided as a roll or a bobbin that is wound around a core so that it may be continuously fed and cut to a piece having an appropriate length for use.

A long probe carrier according to the invention can be manufactured in such a way that a means for applying liquid containing probes is driven to move longitudinally relative to the probe carrier, actually applying the liquid to the carrier. A liquid-ejecting apparatus adapted to eject liquid from any of various types of pipette or nozzle may be used as means for applying liquid for the purpose of the invention.

FIG. 2 is a schematic illustration of a step of operation of applying liquid containing probes onto a string-shaped substrate from a liquid-ejecting apparatus. Referring to FIG. 2, the liquid-ejecting apparatus 3 has nozzle openings 3a-1 through 3a-n for ejecting respective liquids containing different probes and the nozzles are arranged linearly in the longitudinal direction of the carrier. If necessary, it may be so arranged that the liquid-ejecting apparatus 3 has a plurality of nozzle rows arranged in a direction perpendicular to the longitudinal direction of the carrier and one of the nozzle rows may be selectively used by moving the nozzle rows in a direction perpendicular to the longitudinal direction of the carrier. With such an arrangement, a large number of probe species can be applied from the nozzles onto the carrier. In the instance of FIG. 2, the carrier 1 is moved in the direction of arrow relative to the liquid-ejecting apparatus 3 and liquid of the first probe is applied onto the carrier from nozzle opening 3a-1 to form a probe-containing liquid adhering region 2-1. Then, liquids containing different probes are sequentially ejected from the nozzle openings 3a-2 through 3a-4 to form respective probe-containing liquid adhering regions 2-2 through 2-4. FIG. 2 shows a state where liquid is ejected from the nozzle opening 3a-4. If necessary, the nozzle openings down to nozzle opening 3a-n may be used to form probe-containing liquid adhering regions down to region 2-n that carry different respective probes. A large number of probe carriers having the same probes may be formed continuously by repeating the above described operation of applying liquid containing a probe.

If a carrier is provided as a piece cut from a long substrate having an appropriate degree of rigidity, it may be moved by an appropriate conveying means such as a belt conveyor and liquids containing different respective probes may be sequentially applied thereto by means of an liquid-ejecting apparatus.

When the probes contained in the liquid applied to the carrier react with and bonded to the carrier, the reaction will be initiated in a state where liquid containing probes is applied to the carrier to form regions where the probes adhere so that regions fixing probes are produced there. If a processing operation for fixing probes to the carrier needs to be conducted, regions fixing probes are produced by way of such a processing operation that the probes contained in the liquid applied to the carrier are fixed to the latter.

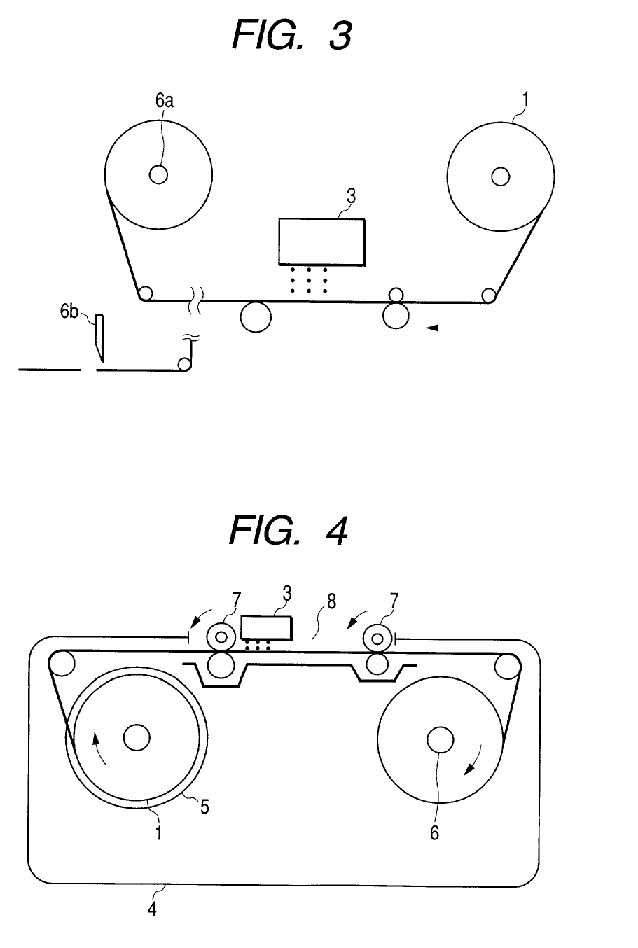

When the carrier is thread-shaped, string-shaped or tape-shaped, the carrier may advantageously be provided in the form of a bobbin or a roll wound around a core so that it may be continuously unwound from the core and fed to the liquid-ejecting apparatus by means of a carrier feeding means formed by combining rollers and guides. FIG. 3 is a schematic illustration of a method of manufacturing a probe carrier by supplying a substrate in the form of a roll. Referring to FIG. 3, thread-shaped carrier 1 that is provided in the form of a roll is unwound and fed to a position where the liquid-ejecting apparatus 3 is arranged by way of a moving path defined by a roller pair, a guide roller and other guide means and liquid containing probes is applied to it there. The thread-shaped carrier 1 may be unwound as the roller pair is driven to rotate by an appropriate drive means. The substrate can be prevented from sagging by applying appropriate backward tension to the roll. Preferably the roll may be provided at the leading end thereof with a dummy portion so that the carrier comes in place when the dummy portion has passed through the location in front of the liquid-ejecting apparatus.

A probe carrier having regions fixing probes that are arranged longitudinally may be provided in the form of a roll or a bobbin wound around a core, or a take-up reel 6a, as shown in FIG. 3 or in the form of a short piece or a chip produced by cutting a long probe carrier by a cutter 6b to an appropriate length.

Alternatively, a probe carrier may be provided in the form of a roll that is contained in a cassette. A probe carrier contained in a cassette will be advantageous when it is fed from the roll and taken up into a roll by a take-up reel after being applied with probes by means of a liquid-ejecting apparatus. FIG. 4 is a schematic illustration of a method of manufacturing a probe carrier by supplying a substrate contained in a cassette that is placed in a probe carrier manufacturing apparatus comprising a liquid-ejecting apparatus. The cassette comprises a cabinet 4, a carrier containing section 5 containing a roll of carrier and a take-up reel 6 along with various guide rollers. The carrier 1 in the form of a roll is unwound and driven to move by drive rollers 7 arranged at a side of the manufacturing apparatus and taken up by the reel 6 that is also driven to rotate. Liquid containing probes is applied to it by the liquid-ejecting apparatus 3 at an opening 8 of the cabinet 4. If necessary, the regions of the carrier 1 where liquid containing probes is applied may be subjected to a drying process and a probe fixing process that are conducted at other respective openings of the cabinet 4 on the way from the opening 8 to the take-up reel 6 from the outside of the cassette.

The probe carrier that is taken up by the reel 6 can be utilized in various forms. For example, the roll of the probe carrier taken up by the reel 6 may be taken out from the cassette and shipped as product. Alternatively, it may be cut into pieces having a predetermined length before shipment. Still alternatively, the cassette containing the roll may be shipped as product. Then, opening 8 of the cassette may be utilized as port for feeding liquid containing a specimen to be analyzed.

If the carrier contained in a cassette is realized in the form of tape and a magnetic recording layer is formed on the surface thereof, data including information on the probes and the results of various observations and examinations may be stored magnetically on the magnetic recording layer.

Now, long probe carriers according to the invention and having any of the above described configurations will be described by way of examples.

### EXAMPLE 1

A paper string with a diameter of 0.1 mm was immersed in a 0.3 mass % agarose solution (heated to 60° C.) and cooled to room temperature to allow the agarose absorbed by the paper string to gel. Subsequently, a predetermined number of SG Clear solutions (aqueous solutions containing glycerol by 7.5 mass %, urea by 7.5 mass %, thiodiglycol by 7.5 mass %, Acetylenol EH (tradename, available from Kawamura Fine Chemicals Co.) by 1 mass %), which were solvents for the bubble-jet printing process, containing respective oligonucleotides having different base sequences as probes at a concentration of 8 μM, were filled separately in the liquid containing sections of a liquid-ejecting apparatus. Then, the paper string was moved along the line of arrangement of the nozzle openings of the liquid-ejecting apparatus and the solutions were applied to predetermined respective positions to obtain a probe carrier having regions that were arranged longitudinally and to which different oligonucleotides were fixed.

### EXAMPLE 2

A paper string with a diameter of 0.1 mm was fed out continuously from a roll and made to pass through an agarose processing tank (containing a 0.3 mass % agarose solution, heated to 60° C.). Then, it was cooled to room temperature to allow the agarose adhering to the paper string to gel. The part of the paper string where agarose was adhering was moved along the line of arrangement of the nozzle openings of a liquid-ejecting apparatus where a predetermined number of SG Clear solutions containing respective oligonucleotides having different base sequences as probes at a concentration of 8 μM were filled separately in the liquid containing sections thereof and the solutions were applied to predetermined respective positions to obtain a probe carrier having regions that were arranged longitudinally and to which different oligonucleotides were fixed.

### EXAMPLE 3

A paper string with a diameter of 0.1 mm was fed out continuously from a roll and moved along the line of arrangement of the nozzle openings of a liquid-ejecting apparatus, while a 0.3 mass % agarose solution (in a molten state) and a predetermined number of SG Clear solutions containing respective oligonucleotides having different base sequences as probes at a concentration of 8 μM were ejected onto predetermined respective positions of the paper string. The liquid-ejecting apparatus comprised a liquid containing section filled with the agarose solution held in a molten state and other liquid containing sections filled with the respective oligonucluotide solutions and was so controlled that the oligonucleotide solutions were applied to the paper string at a stage where the agarose solution had been applied to the paper string and the agarose adhering to the paper string had gelled.

Note that, in an operation of applying liquid to the paper string, liquid is preferably applied to the paper string while the latter is axially rotated. Then liquid may be applied to the entire peripheral surface of each of the predetermined regions of a paper string so that the liquid of each region may not be mingled with those of the adjacent regions and that each of the probes and the corresponding target substance in the specimen may contact with each other effectively and efficiently.

### EXAMPLE 4

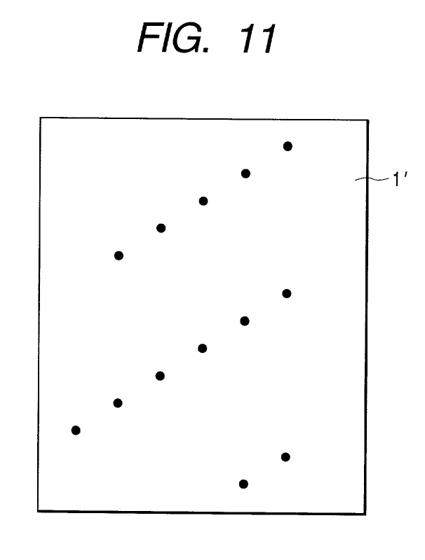

Oligonucleotides were fixed to a nitrocellulose membrane as probe DNAs in such a manner that they would be arranged in axial row when the membrane was rolled to form a hollow cylinder as shown in FIG. 11. Then, the membrane was wound around a rod with a diameter of 0.1 mm with the regions fixing the probe DNAs facing inside and secured to the rod. The membrane was then heated to 180° C. to fix the DNAs to the rod and obtain a rod-shaped probe carrier chip to which DNAs were bonded.

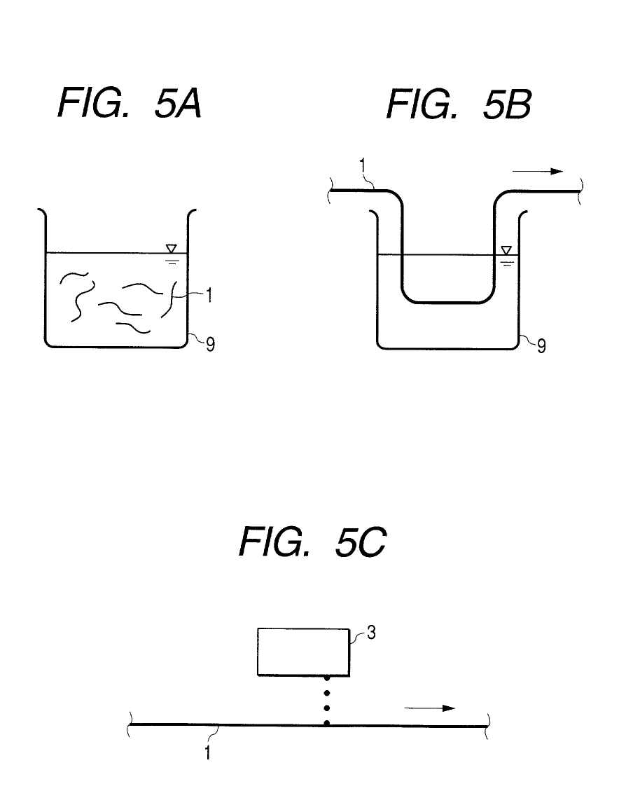

Techniques for applying a specimen to a long probe carrier may include a method of immersing a long probe carrier 1 into liquid 9 containing the specimen (see, inter alia, FIGS. 5A and 5B) and a method of applying liquid containing a specimen to a probe carrier 1 by means of a liquid-ejecting apparatus 3 (see, inter alia, FIG. 5C).

The reaction of the target substance contained in a specimen and the probes fixed to a carrier can be detected by signalizing it by means of an appropriate method that can be selected from a number of methods. A popular method may be the use of a label substance, which may be a fluorescent substance that emits an optical signal that can be detected by a sensor.

For instance, when the probe carrier and the specimen are brought into contact with each other and are labeled with a fluorescent substance to take out the reaction of the target substance and the probes as fluorescent light, presence or absence of the reaction of the probe and the target substance in each of the regions fixing probes can be detected by moving the long probe carrier relative to the detecting means such as a sensor.

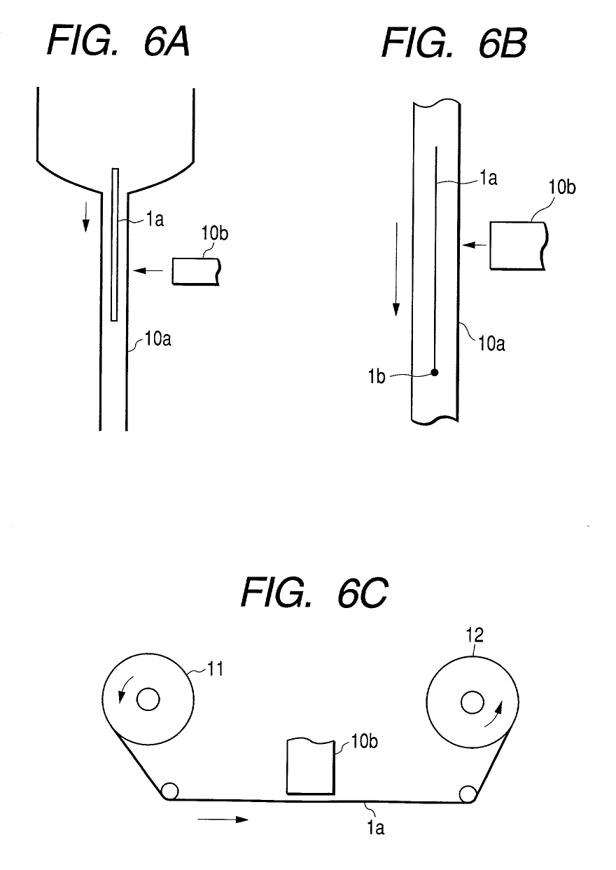

For example, when the probe carrier is realized in the form of a rigid and long chip, the path in an appropriate tube 10a as shown in FIG. 6A may be used. Then, the reactions of the probes and the target substance can be successively detected if the probe carrier 1a (to be referred to as sample for observation hereinafter) is held in a state where it can be brought into contact with a specimen and the reactions of the probes and the target substance can be taken out as fluorescent light and moved through a detection zone of a detection means 10b continuously from the leading end thereof. It is also possible to control the rate at which the sample for observation moves through the detection zone by flowing liquid that does not influence the sample to be observed through the tube 10a.

If a thread-shaped or string-shaped probe carrier as shown in FIG. 6B is used, it is possible to make the sample for observation la to pass through a detection zone where a detection means 10b is arranged by providing the leading end of the sample for observation with a weight 1b during the observation period and allowing the sample for observation 1a to move forward through the detection zone.

In the instances of FIGS. 6A and 6B, it is preferable that the probes are fixed to the probe carrier by utilizing the entire peripheral surface of the latter, considering that the thread-shaped or string-shaped sample for observation la can be rotated around the longitudinal axis thereof.

On the other hand, if the sample for observation la needs to be collected in the form of a bobbin or a roll for storage, it may be fed out from a feeding roll 11, driven to pass through a detection zone where a detection means 10b is arranged and taken up for collection by a take-up reel 12 as shown in FIG. 6C. This arrangement is advantageous in that, if the sample for observation is tape-shaped and probes are fixed to one of the opposite surfaces thereof, the surface carrying the probes fixed thereto can be aligned in a direction good for the detecting operation of the detection means because the feeding direction of the tape-shaped sample for observation can be controlled typically by means of conveyance guides.

Alternatively, a cassette as shown in FIG. 4 may be used in a manner such that the probe carrier and the specimen are held in contact with each other in advance to obtain a roll of sample for observation which is in a state where presence or absence of a reaction of each probe and the target substance can be detected. Then, the roll of sample for observation is set in the roll containing section 5 of the cassette and is taken up by a take-up reel 6 at timings necessary for observation and presence or absence of a reaction to be detected by the detection means that may be a sensor is optically determined by utilizing an opening 8.

The detection apparatus that is used with a cassette may comprise a fitting section of the cassette including a roll containing section for containing a roll obtained by taking up the sample for observation on a feed reel as described earlier, a take-up reel for taking up the sample for observation fed out from the feed reel and an opening arranged along the moving path of the sample for observation extending between the roll containing section and the take-up reel, a drive means for driving the take-up reel of the cassette arranged in the fitting section and an aligning section for aligning the signal detection means of the detection apparatus to a position where it can detect the signal from a region fixing a probe of the sample for observation that is being driven to move on the moving path by the drive means by way of the opening.

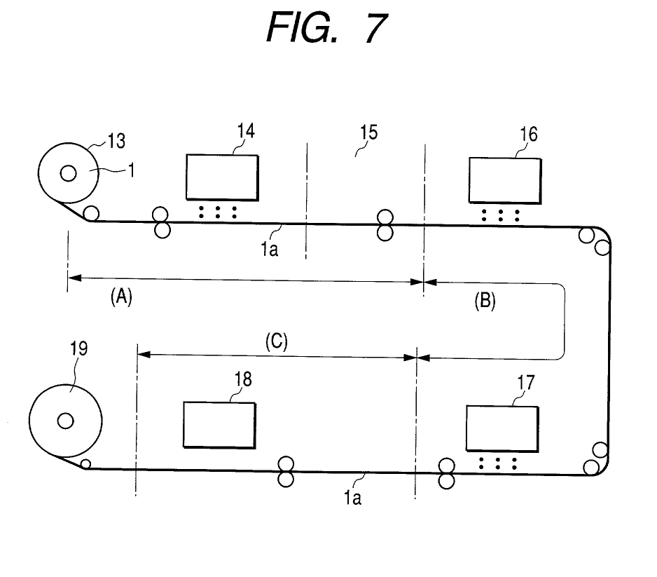

When the carrier is provided in the form of a bobbin or a roll, an examination system as shown in FIG. 7 may be used. The examination system comprises a carrier containing section 13 for containing a bobbin-shaped or roll-shaped carrier 1, a probe applying section 14 for applying liquid containing probes to the carrier la from a liquid-ejecting apparatus, a probe carrier finishing section 15 for performing a drying operation and a processing operation for fixing probes to the probe carrier, a specimen applying section 16 for applying a specimen to be analyzed to the probe carrier, a label processing section 17 for processing the signal from the label into a detectable form and a detection section 18 for sensing a signal from the label and detecting presence or absence of a reaction of a probe and the target substance in the specimen in the corresponding region fixing the probe on the probe carrier. Referring to FIG. 7, it is also possible to unitize part A for manufacturing a probe carrier, part B for applying a specimen and producing a state where the corresponding label can function and part C for detecting the signal from the label. If necessary, two or more than two of these parts may be collectively unitized.

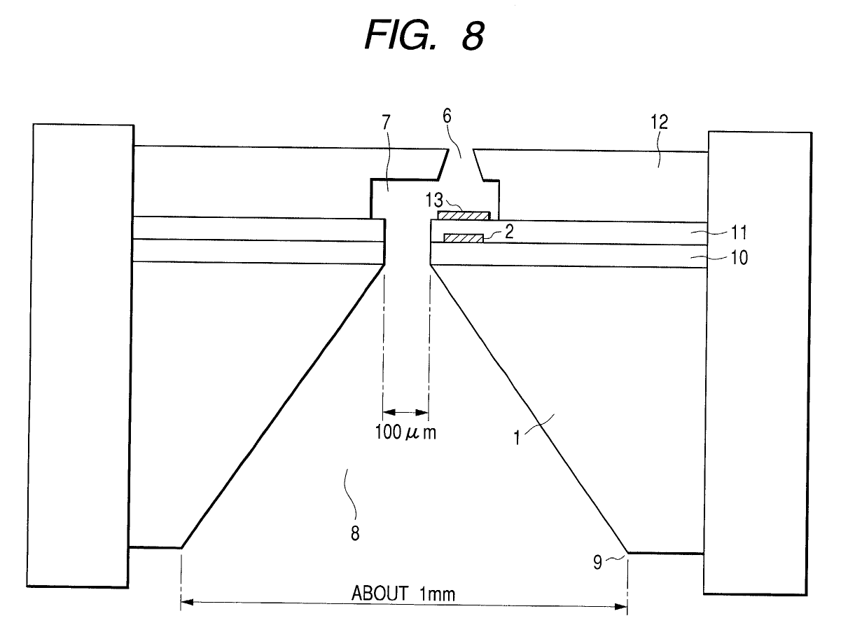

When a liquid-ejecting apparatus is used for applying liquid containing probes and a specimen to a carrier, it is advantageous that the liquid-ejecting apparatus comprises one or more than one liquid containing sections and one or more than one liquid ejecting sections, each including a nozzle connected to the corresponding liquid containing section and adapted to eject liquid, and liquid ejection energy generating means for generating energy necessary for ejecting liquid from the nozzles. The number of liquid containing sections and that of liquid ejecting sections may be selected appropriately depending on the types of liquid to be ejected. While the liquid ejection energy generating means may be any of a variety of types including the piezoelectric type and the heating type, heating elements may be advantageously be used for generating thermal energy to cause film-boiling of the liquid and ejecting the liquid through the openings under pressure of the boiling liquid, particularly when a large number of liquid ejecting sections need to be densely and independently provided. Such a liquid-ejecting apparatus preferably has a structure where bubbles generated by film-boiling can communicate with external air by way of nozzle openings. FIG. 8 illustrates the liquid ejecting section of such a liquid-ejecting apparatus.

Referring to FIG. 8, there are shown an insulating film 10, a protection film 11, a nozzle carrying member 12 and an anti-cavitation film 13 typically made of Ta. The insulating film 10 may be a thermal oxide film formed by thermally oxidizing a silicon substrate, an oxide film formed by CVD, a nitride film also formed by CVD or some other films. The protection film 11 may be an oxide film formed by CVD, a nitride film also formed by CVD or some other films. The nozzle carrying member 12 constituting a nozzle 6 and a flow path 7 may be formed by applying a nozzle member where the nozzle and the flow path are produced in advance to a semiconductor substrate or in a similar manner to a semiconductor process utilizing the photolithography technology.

Particularly, when the liquid-ejecting apparatus needs to be prepared to occupy a large area, problems such as creases, warping and displacement can take place if the nozzle carrying member provided with nozzles and flow paths is bonded to a substrate because the nozzle carrying member also has a large area so that the chip preparing operation can be a difficult one. Therefore, the use of a process of forming nozzles by laying a nozzle forming layer on a substrate by means of a photolithography technique is preferable. For example, Japanese Patent Application Laid-open No. 62-264957 describes a nozzle forming process using photolithography.

Referring back to FIG. 8, supply port 8 is produced by anisotropic etching of silicon and opened at an angle of 54.7° relative to the substrate surface as shown in FIG. 8. The supply port shows a profile of a frustum of quadrangular pyramid. For instance, as a silicon wafer is immersed in an etching solution such as a 22 mass % solution of TMAH (tetramethylammonium hydroxide) for anisotropic etching, etching proceeds in the areas of the opposite surfaces of the wafer where silicon is exposed to produce the profile of the supply port and eventually the etched parts of the opposite sides of the wafer come across to produce a through hole.

While the major role of the supply port of a conventional printing ink-jet head is to lead ink from the connecting section of the head to the ink tank that is connected to the rear surface of the substrate to the heater section, the supply port of a liquid-ejecting apparatus for manufacturing a probe carrier can be used as liquid reservoir because the total amount of liquid to be ejected is small.

Liquid is led from the supply port 8 at the rear surface of the substrate to the front surface of the substrate and then to the nozzle 6 by way of the flow path 7. In other words, the flow path that runs through the substrate is formed by the supply port (liquid reservoir), the flow path and the nozzle.

As a voltage is applied to the opposite ends of the heater by way of electrode wires (not shown), the liquid located close to the heater is superheated to cause film bubbling and liquid is ejected.

Thus, a liquid-ejecting apparatus can be formed by arranging a required number of liquid ejecting sections, each having a configuration as described above, linearly or on a plane.

A liquid-ejecting apparatus and an apparatus for manufacturing a probe carrier that comprises a liquid-ejecting apparatus according to the invention may be prepared by employing such components that are used in an ink-jet recording system to be used for printing, a head to be used with such a system or a recording apparatus to be used also with such a system and selected according to the application of the liquid-ejecting apparatus. Additionally, any of the components may be modified appropriately according to the application of the liquid-ejecting apparatus. An example of utilization of such an inkjet recording system may be a recording head and a recording apparatus comprising a means for generating thermal energy such as an electrothermal converter or a laser device to be utilized for ejection of ink and adapted to change the state of ink by such thermal energy. An ink-jet recording system having such a configuration provides remarkable advantages for the purpose of the invention because it can realize high density and high definition recording.

The basic configuration and the principle of, for example, U.S. Pat. No. 4,723,129 or those of U. S. Pat. No. 4,740,796 may advantageously be utilized for the purpose of the invention. While the principles of these patent documents are applicable to both on-demand type liquid-ejecting apparatus and continuous type liquid-ejecting apparatus, the use of the on-demand type is particularly advantageous for the purpose of the invention because at least a drive signal corresponding to the information to be recorded and adapted to produce a rapid temperature rise exceeding nucleus boiling is applied to an electrothermal converter arranged in correspondence to a sheet or a liquid path holding liquid (ink) so as to generate thermal energy in the electrothermal converter and produce film boiling on the thermally acting plane of the recording head. Then, as a result, a bubble that shows a one-to-one correspondence to the drive signal is formed in the liquid (ink). Liquid (ink) is ejected through the ejection opening to produce at least a droplet as a result of the growth and contraction of the bubble. The growth and contraction of a bubble can be realized immediately and appropriately to eject liquid (ink) with an enhanced level of responsiveness particularly when a pulse signal is used for the drive signal. A pulse-shaped drive signal as described in either of U.S. Pat. Nos. 4,463,359 and 4,345,262 may suitably be used for the purpose of the invention. The recording effect can be further improved by using the conditions described in U.S. Pat. No. 4,313,124 that discloses an invention on the temperature rise ratio of a thermally acting plane.

Beside the combined use of an ejection port, a liquid path and an electrothermal converter (linear liquid flow path or rectangular liquid flow path) as disclosed in any of the above cited patent documents, the use of a curved region for arranging a thermally acting section as disclosed in either of U.S. Pat. Nos. 4,558,333 and 4,459,600 is within the scope of the present invention. Additionally, the use of a common slit for the ejecting section of a plurality of electrothermal converters as disclosed in Japanese Patent Application Laid-open No.59123670 and the arrangement of juxtaposing an open hole for absorbing pressure waves of thermal energy and the ejecting section as disclosed in Japanese Patent Application Laid-open No.59-138461 can also advantageously be used for the purpose of the present invention. In short, regardless of the form of the recording head, a recording operation can be conducted reliably and efficiently according to the invention.

The present invention is also effectively applicable to a full-line type recording head having a length corresponding to the largest width of the recording medium on which a recording apparatus can record information. Such a recording may be realized either by combining a plurality of recording heads in order to adapt itself to the necessary length or by using an integrally formed single recording head.

Furthermore, the present invention is equally effective when a serial type recording head that is rigidly secured to the apparatus main body, a chip type recording head that is replaceable and adapted to be electrically connected to and supplied with ink from the apparatus main body as it is mounted on the apparatus main body or a cartridge type recording head integrally formed with an ink tank is used.

Preferably, the recording apparatus to be used for the purpose of the invention is additionally provided with an ejection recovery means and an auxiliary spare means of the recording head for the purpose of improving the stability of operation. Specific examples of such means include a recording head capping means, a cleaning means, a pressurizing or suction means, a preliminary heating means using an electrothermal converter, a separate heating element or a combination thereof and a spare ejection means to be used for purposes other than recording.

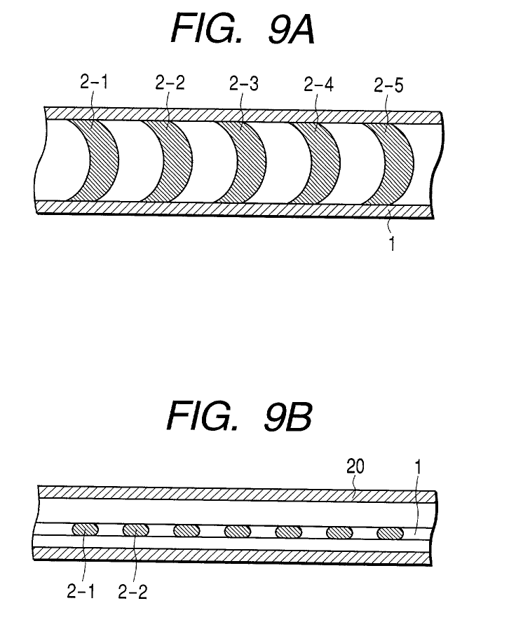

The use of a film boiling system is very effective for ejecting ink for the purpose of the invention. (Tubular probe carrier and utilization thereof) In another embodiment of probe carrier according to the invention, regions fixing different probes are arranged in a hollow section of a tubular member located inside of the tubular member, at least part of which transmits light, along the axial direction of the tubular member. The regions fixing probes in the inside of the tubular member may be arranged on the inner wall of the tubular member. Alternatively, they may be produced by inserting a long probe carrier as described earlier into the hollow section of the tubular member. FIG. 9A illustrates regions 2-1 through 2-5 fixing probes of a probe carrier arranged on the inner surface of a tubular member in the axial direction thereof and FIG. 9B illustrates a long probe carrier inserted into a tubular member.

Such a tubular probe carrier can be manufactured by any of the manufacturing methods listed below.

(1) Regions fixing probes are formed by applying liquid containing probes onto a light transmitting substrate that can be worked to show a tubular profile and the substrate is then actually worked into a tubular form.

(2) Regions fixing probes are formed by applying liquid containing probes onto a plate-shaped light transmitting substrate and the substrate is then warped into a tubular form.

(3) Grooves are formed on the surface of a plate-shaped or tape-shaped substrate and regions fixing probes are formed in the grooves. Thereafter, the grooved surface is covered by a light transmitting cover and a tubular probe carrier is formed from the substrate.

With the method of (1), regions where different probes are fixed are arranged at respective predetermined positions of a glass substrate that can be molten and worked at temperatures where DNAs can be left on a stable basis. Then, the substrate is warped into a tubular form to produce a probe carrier.

With the method of (2), regions fixing probes are formed on the surface of a plate-shaped substrate according to the designed arrangement of the regions on the surface of the substrate when the latter is warped into a tubular form. Then, the substrate is warped into the form of a roll to produce a probe carrier. For example, oligonucleotides are applied as probe DNAs onto a nitrocellulose membrane as shown in FIG. 11. Subsequently, the membrane is wound around the outer peripheral surface of a rod having a diameter of 0.1 mm with the regions fixing the probe DNAs facing the inside. Then, the membrane is rigidly held to keep its shape. Thereafter, the membrane is heated to 180° C. to fix the DNAs onto the rod to produce a rod-shaped probe carrier chip to which DNAs are bonded.

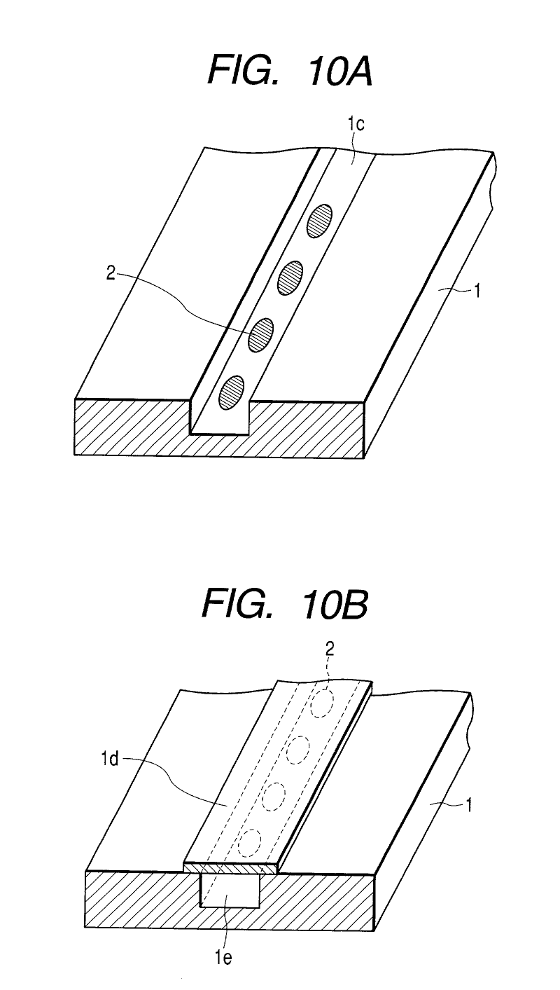

With the method of (3), one or more than one grooves are formed on a predetermined surface of a plate-shaped appropriate substrate and regions 2 fixing probes are arranged there as shown in FIGS. 10A and 10B. Subsequently, lid members 1d are bonded to the substrate to form hollow sections le of a tubular member.

Note that with the method of (3) of forming grooves in a substrate and subsequently covering them by means of lids, the lids are made of a light transmitting material. Therefore, the substrate may be light-transmitting or not light-transmitting. However, when a signal generated on the basis of presence or absence of a reaction of a probe arranged in an area other than the covers with a target substance is to be detected, the substrate is preferably made of a light-transmitting material.

Light-transmitting materials that can be used for the substrate include resin and glass of various types.

The inner diameter of the tubular member may be selected appropriately depending on the application of the probe carrier. The internal volume can be reduced to by turn reduce the necessary amount of the specimen when the inner diameter is less than 1 mm.

The cross section of the inner wall taken along a direction perpendicular to the axial direction of the tubular probe carrier is not limited to circular and may show any appropriate form.

When presence or absence of the target substance is detected by using a tubular probe carrier having a configuration as described above, the specimen is typically brought into contact with the regions for fixing probes by feeding liquid containing the specimen into the tube of the tubular member by means of a pump. Additionally, labels are applied and a cleaning operation is conducted by sequentially feeding respective liquids containing ingredients necessary for the operation into the tubular member. Then, the optical signal transmitted from the inside of the tubular member to the outside by way of the light transmitting part of the tubular member is detected and presence or absence of the target substance in the specimen is determined by moving the detection means relative to the tubular member along the axial direction of the tubular member and utilizing labels.

When such a tubular member is used, the consumption of liquid containing the specimen and that of reagents necessary for various processing operations can be remarkably reduced by reducing the inner diameter of the tubular member. Additionally, the signal from the label can be read by linearly moving at least either the detection means or the tubular probe carrier to remarkably improve the efficiency of the detecting operation.

What is claimed is:

1. A probe carrier having on an elongate substrate a plurality regions fixing different probes each adapted to be bonded specifically to a target substance, said regions fixing probes being arranged longitudinally on said substrate.

2. A probe carrier according to claim 1, wherein
said substrate is string-shaped, tape-shaped, string-shaped, tube-shaped or rod-shaped.

3. A probe carrier according to claim 1, wherein
said target substance is a nucleic acid and said probe is a single stranded nucleic acid having a base sequence complementary to all or part of the base sequence of the target nucleic acid and adapted to be specifically hybridized with the target nucleic acid.

4. A probe carrier roll formed by winding on a core a probe carrier having on an elongate substrate a plurality regions fixing different probes each adapted to be bonded specifically to a target substance, said regions fixing probes being arranged longitudinally on said substrate.

5. A cassette for detecting a target substance by means of probes adapted to be bonded specifically to the target substance, said cassette comprising in a cabinet:
a probe carrier roll containing section containing a probe carrier roll formed by winding on a feed reel a probe carrier having on an elongate substrate a plurality regions fixing different probes each adapted to be bonded specifically to a target substance, said regions fixing probes being arranged longitudinally on said substrate;
a take-up reel for taking up the probe carrier fed out from the feed reel; and
an opening arranged on the moving path of the probe carrier from said probe carrier roll containing section to said take-up reel.

6. A method for detecting a target substance in a specimen by means of probes adapted to be bonded specifically to the target substance, said method comprising:
a step of causing a probe carrier having regions fixing different probes to contact with the specimen, the regions being arranged in the longitudinal direction of an elongate substrate; and
a step of detecting presence or absence of a reaction of the target substance with the probe of each of the regions fixing probes caused to contact with the specimen.

7. A method according to claim 6, wherein
the reaction of the target substance with the probes is taken out as a signal detectable by a detection means and the signal is read by moving the regions fixing probes brought into contact with the specimen relative to the detection means.

8. A method according to claim 6, wherein
said probe carrier is brought into contact with the specimen by moving a liquid-ejecting apparatus for applying the specimen relative to the probe carrier in the longitudinal direction of the latter and applying a solution of the specimen to each of the regions fixing probes from the liquid-ejecting apparatus.

9. A method according to claim 8, wherein
said probe carrier is supplied in the form of a roll wound on a core and the probe carrier fed from the roll is brought into contact with the solution of the specimen.

10. A method according to claim 9, wherein
said probe carrier is supplied in the form of a cassette having in a cabinet a probe carrier roll containing section containing a probe carrier roll formed by winding the probe carrier on a feed reel, a take-up reel for taking up the probe carrier fed from the feed reel and a specimen applying opening arranged on the moving path of the probe carrier extending between the probe carrier roll containing section and the take-up reel, and the solution of said specimen is applied to each region fixing a probe by way of the specimen applying opening.

11. A method according to claim 6, wherein
said substrate is string-shaped, tape-shaped, string-shaped, tube-shaped or rod-shaped.

12. A target substance detecting apparatus for detecting presence or absence of a reaction of probes with a target substance in a specimen by detecting a signal generated by the reaction, the probes being adapted to be bonded specifically to the target substance, said apparatus comprising:
a signal detecting means; and
a moving means for longitudinally moving a sample for observation relative to said detecting means, said sample for observation being obtained by causing the specimen to contact with a probe carrier having regions fixing different probes, said regions being arranged in the longitudinal direction of an elongate substrate.

13. An apparatus according to claim 12, wherein
said moving means is adapted to sequentially move the sample for observation relative to said detecting means from the leading end toward the rear end thereof.

14. An apparatus according to claim 13, further comprising:
a roll containing section containing a roll formed by winding the sample for observation on a feed reel, a take-up reel for taking up the sample for observation as fed out from the feed reel, a take-up reel drive means and a detecting section including said detecting means and arranged on the moving path of the sample for observation extending between the roll containing section and the take-up reel.

15. An apparatus according to claim 14 further comprising:
a cassette receiving section for receiving a cassette having in a cabinet a roll containing section for containing a roll formed by winding the sample for observation around a feed reel, a take-up reel for taking up the sample for observation as fed out from the feed reel and an opening arranged on the moving path of the sample for observation extending between the roll containing section and the take-up reel, a take-up reel drive means for driving the take-up reel of the cassette received in the cassette receiving section and an aligning means for aligning said detecting means at a position adapted to detect by way of the opening the signal from the regions fixing probes of the sample for observation moving along the moving path as driven by said drive means.

16. An apparatus according to claim 12, wherein
said substrate is string-shaped, tape-shaped, string-shaped, tube-shaped or rod-shaped.

17. A method for detecting a target substance in a specimen by means of probes adapted to be bonded specifically to the target substance, said method comprising:
(1) a step of preparing a probe carrier by arranging regions fixing different probes in the longitudinal direction of an elongate substrate;
(2) a step of obtaining a sample for observation by causing the probe carrier to contact with the specimen: and
(3) a step of detecting presence or absence of a reaction of the target substance with the probe in each of the regions fixing probes in the sample for observation.

18. A method according to claim 17, wherein
the reaction of the target substance and the probes is detected by a detecting means as a detectable signal and the signal is read by moving the regions fixing probes held in contact with the specimen relative to the signal detecting means.

19. A method according to claim 17, wherein
said probe carrier is brought into contact with the specimen by moving a liquid-ejecting apparatus for applying the specimen relative to the probe carrier in the longitudinal direction thereof and applying the liquid of said specimen to each of the regions fixing probes from the liquid-ejecting apparatus.

20. A method according to claim 17, wherein
said probe carrier is supplied as a roll formed by winding the probe carrier around a core and the probe carrier is brought into contact with the solution of said specimen as it is fed out from the roll.

21. A method according to claim 20, wherein
said probe carrier is supplied as a cassette having in a cabinet a probe carrier roll containing section for containing a roll formed by winding the probe carrier around a feed reel, a take-up reel for taking up the probe carrier as fed out from the feed reel and a specimen applying opening arranged on the moving path of the probe carrier extending between the roll containing section and the take-up reel and the solution of said specimen is applied to each of the regions fixing probes by utilizing the specimen applying opening.

22. A method according to claim 21, wherein
said cassette has an opening to be used for detecting the signal on the moving path of the probe carrier between said specimen applying opening and said take-up reel and the signal is detected by using said opening.

23. A method according to claim 17, wherein
said substrate is string-shaped, tape-shaped, string-shaped, tube-shaped or rod-shaped.

24. A detection apparatus for detecting a reaction of probes adapted to be bonded specifically to a target substance with the target substance in a specimen by detecting presence or absence of a signal generated by the reaction, said apparatus comprising:
a probe carrier preparing section for preparing a probe carrier carrying probes fixed thereto on an elongate substrate;
a sample preparing section for preparing a sample for observation by causing the probe carrier prepared by said probe carrier preparing section to contact with a specimen; and
a detecting section for detecting a signal generated by the reaction of the probes with a target substance in the sample for observation as prepared by said sample preparing section;
said probe carrier preparing section having:
a means for applying solutions of the probes;
a means for holding the substrate; and
a first moving means for moving said solution applying means longitudinally relative to the substrate;
said detecting section having:
a means for detecting a signal; and
a second moving means for moving said signal detecting means relative to the sample for observation as prepared by causing the probe carrier having regions fixing different probes to contact with the specimen, said regions being arranged in the longitudinal direction of the substrate.

25. An apparatus according to claim 24, wherein
said second moving means is adapted to sequentially move the sample for observation relative to said detecting means from the leading end toward the rear end thereof.

26. An apparatus according to claim 24, further comprising:
a roll containing section containing a roll formed by winding the substrate around a feed reel, a take-up reel for taking up the substrate as fed out from the feed reel, a take-up reel drive means and a guide mechanism adapted to sequentially move said probe carrier preparing section, said specimen preparing section and said detecting section by the operation of taking up the substrate fed out from the containing section by means of the take-up reel.

27. An apparatus according to claim 24, wherein
said substrate is string-shaped, tape-shaped, string-shaped, tube-shaped or rod-shaped.

28. A method for manufacturing a probe carrier having regions for fixing different probes adapted to be bonded specifically to a target substance, said regions being arranged in the longitudinal direction of an elongate substrate, said method comprising:
a step of moving a means for applying probe solutions relative to said substrate from the front end to the rear end thereof so as to apply different probe solutions as a function of the arrangement of said regions; and
a step of fixing the probes in said regions carrying the respective probe solutions applied thereto to said substrate.

29. A method according to claim 28, wherein
said substrate is supplied as a roll formed by winding the substrate around a reel and said different probe solutions are applied to the substrate by moving the application means relative to the substrate fed out from the roll.

30. A method according to claim 29, wherein
said roll is formed by winding a probe carrier formed by fixing the probes onto the substrate around a feed reel.

31. A method according to claim 28, wherein
said means for applying probe solutions has nozzles for ejecting the respective solutions and the solutions are ejected from the nozzles by heat applied form a thermal energy generator.

32. A probe carrier manufacturing apparatus for 4 manufacturing a probe carrier having regions fixing different probes adapted to be bonded specifically to a target substance, said regions being arranged in the longitudinal direction of an elongate substrate, said apparatus comprising:
a means for applying probe solutions; and
a means for relatively moving said solution applying means from the front end to the rear end of the long substrate.

33. An apparatus according to claim 32, further comprising
a roll containing section for containing a roll formed by winding the substrate around a feed reel and said solution applying means is moved relative to the substrate fed out from the roll by said moving means.

34. An apparatus according to claim 33, further comprising:
a cutting means for cutting the probe carrier formed by fixing the probes onto the substrate into pieces having a predetermined length.

35. An apparatus according to claim 34, further comprising:
a take-up reel for taking up the probe carrier formed by fixing the probes onto the substrate and collecting it and a take-up reel drive means.

36. A probe carrier comprising:
a plurality of regions fixing probes arranged axially in the inside of a tubular member, said regions carrying different probes fixed thereto.

37. A method for detecting a target substance in a specimen by means of probes adapted to be bonded specifically to a target substance, said method comprising:
a step of flowing a solution containing a specimen from one of two openings of a tubular member as a probe carrier to the other opening, a plurality of regions fixing probes being arranged axially in the inside of the tubular member, said regions carrying different probes fixed thereto; and
a step of detecting presence or absence of a reaction of the target substance with the probe of each of said regions fixing probes caused to contact with the specimen.

38. A method of manufacturing a probe carrier having a plurality of regions fixing probes adapted to be bonded specifically to a target substance, said regions being arranged axially in the inside of a tubular member, said regions carrying different probes fixed thereto, said method comprising:
a step of causing solutions of a plurality of probes necessary for forming and arranging the regions to adhere to a plate-shaped member according to the arrangement of the regions;
a step of solidifying the probes in the regions carrying the probes adhered thereto on the plateshaped member; and
a step of molding the plate-shaped member carrying the solidified probes adhered thereto into a tubular member so as to make the regions fixing probes to be arranged in the axial direction on the inner wall of the tubular member.

39. A method for detecting a target substance in a specimen by means of probes adapted to be bonded specifically to a target substance, said method comprising:
a step of flowing a solution containing the specimen into the inside of a tubular member having regions fixing probes arranged in the axial direction in the inside thereof and causing the solution to contact with each of the regions fixing probes to obtain a sample for observation; and
a step of detecting presence or absence of a reaction of the target substance with the probe of each of the regions fixing probes in the sample for observation.

40. A method according to claim 39, wherein
the reaction of the target substance and the probes is detected by a detecting means as a detectable signal and the signal is read by moving the regions fixing probes held in contact with the specimen relative to the signal detecting means.
